SEQUENCE LISTING (2 sequences)

SEQ ID NO: 1 - DNA, 4664 bp, Homo sapiens
gcttgctcccaatcacaggagaaggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaaaaaaaaaaaaaaaaaa

SEQ ID NO: 2 - DNA, 5336 bp, Homo sapiens
cggagggggaggtggctgccgcttctcccgcgtccgccattttgttgctgtggctattgggaacaagctgggcaaaagcaccccggaggcgcgacgctccttcgagttcggtgcctcgtgtgacggcgggggtcggtgaagacccgtcgagctgcggcgccggcgcgttccaggccgggagtcactggaggcacccctgggacgccgagcagcccgagaaccccggggtggcctccgctgcggctcgggtttgcctgccccgaccccccggctctgccgtgcattcccgggcggctctctccgtgtggcggccccggagcaggcgggcggcgtcggaggatgctgcgggcccggagccgagaggaaagtgctggcccagccctctgagcgctcctcgaggtgtgcgagaggcccttcctcggccccaaagccgtctgccgggctaaggcgtgcagagcaggcgaggacagccgccgcccctaccgccgcagagtccccggtccaacaccatgtcctccgccaggttcgactcctcggaccgctccgcctggtatatggggccggtgtctcgccaggaggcgcagacccggctccagggccagcgccacggtatgttcctcgtccgcgattcttccacctgccctggggactatgtgctgtcggtgtccgagaactcgcgggtctcccactacatcatcaactcgctgcccaaccgccgttttaagatcggggaccaggaatttgaccatttgccggccctgctggagttttacaagatccactacctggacaccaccaccctcatcgagcctgcgcccaggtatccaagcccaccaatgggatctgtctcagcacccaacctgcctacagcagaagataacctggaatatgtacggactctgtatgattttcctgggaatgatgccgaagacctgccctttaaaaagggtgagatcctagtgataatagagaagcctgaagaacagtggtggagtgcccggaacaaggatggccgggttgggatgattcctgtcccttatgtcgaaaagcttgtgagatcctcaccacacggaaagcatggaaataggaattccaacagttatgggatcccagaacctgctcatgcatacgctcaacctcagaccacaactcctctacctgcagtttccggttctcctggggcagcaatcacccctttgccatccacacagaatggacctgtctttgcgaaagcaatccagaaaagagtaccctgtgcttatgacaagactgccttggcattagaggttggtgacatcgtgaaagtcacaaggatgaatataaatggccagtgggaaggcgaagtgaacgggcgcaaagggcttttcccctttacgcacgtcaaaatctttgaccctcaaaacccagatgaaaacgagtgattgctgttgccctgtttcctgctgctttgttgttctgcctgtcctagtctcctttgaagtgggaaagcattttctctcataggcaagtcacactgcattgccgaagtccagctttctgcagactggcagtcgcacacacatttggaatgcacacagcggctgcctcctgatgtttgtatcatagtcgtattgtcaaagagtagccgattttagagttcttttggatcataaactggaaatactgatggaagcacacaagtggagagaagttgacatggaaagggtcttccttctcattgctgcccgtttgtacatgggactgattctgttgtgttcaccagagaaagcttgaggccatggcgagatactgcatgtttgctgttccacaaagcagtggcttagctgccatcttgcttttctttggacaacaggaagtgaaccttaaggaagagagaattctgttctaaaactccaaaatctggcttttttttttcttttgttttggtttggttttggaaaactaattaattagacttgtgtgggggtttttttttgttttgttttgtttgttttgttttgtttttttgagacggtgtctcgctgcatcacccaggctggagtgcagtggcgcaatctcgccttcctgcagtctccgcctcctgagttcaagcgattctcctgcctcagcctcccaagtagctgggactgcaggcgcgcacaccacgcccagctaatttttgtatttttagtagagacggggtttcatcatgttgaccaggctggtctcaaactcctgacttcaggtgatccacccgccttcagcctcccaacgtgctgggattacagccgtgagccgccgcgcctggcctagacttgtgtgttctaaacaggttaagtagcaggttgggtttttatgatagtgcaaggaatgactcatgcctctgagcttctaaactgaagctgctgtaactaaaggaatctgaaaagaacaaccctgaagcagaggcctttattgtcttggttgccagtagtaccttgttttgccatgtagcagacaacacacaaaataatgcagttgtggtgtgccatgctatgtgcacagccccttggattactttgttttaaaaagcatcagagttgggggtactttagggaaacctttgcttaccttgttttgccagtgataagagcagtgggttggagggcacttggccagttttctgttcagcttttcagtgaatgtacccctttaaggttcagacttaaacttccttaaaaagtggcgttgttcatagaatcgttggactcattaatgaatcgttcaactccactcactgaagcccagacctccgtgcccaggcccaatctcgtcaggctgccagagaaagttggtgctgctcatactggtctcacagtctaagtaagtgtctgtgatgctcccaagcaaaggaaatgcaagctctggaaattcgttaatgtatttgatgtcttagtgttttagtgactagggagaccattaactagtttatcattaaccacttatcagtgtattgatgttaaagcatttccctgttagctaaaagaggcctgttcatacaagccaactggtatatacgtgtggttcatccatcatctgctgcacatagcagactagaattctgggaaccctgtgcaattcagtctgctctcccttgtggaccctggtaaagaaaagcctcagctcatagtgaacacagcagacctagaaatgtagcagcagcctactgagtagctttcatttactgatcatctgctgtgactgtggccctgtctggaggttcctaggttttgagatttagagcaatgcattctggagacagaaccagcagaacagccatttttcaatttttctttaaatcagtattccatcaggcagataactgctgtattcatgaatcttgagagtgttcctgagacagaattaatggtcatttgggaaaactatcgccatggcttcccatctgtggttttcctctaaaagccttggagattagcccttccttgccagtgagaacggtgaccgcctcctgctctgcacggtctgcggcagttgccgcttctggttaggtgtgtcaggttggcttattttgggttcaggcctggcgtagcacccacaagtggcagacatatcacaagagtccccagactctgcctagaaacagtgtttgccctttggccagtgacgtggttcatcccggcccatgttgagccatgagtggagtttccaacagagggaggaatgtgtgccttgttcaaggagggcacgacccttaggcctttttcaaccagatttagctgaagggcttgacacctttgaattacagcagttgactcagagtgcaagaagtctggccattttggaaagcaaggtttcctttcagccctgtctactgaccaataccccgactcaccttgtgtggcgcacttcagaatcagatatacctagagtatacctgtggtttggttttataattaatcagctcgttacttcagcccatgaaaatggcatccagggctgccaggagattcagagctcaaaacaaggcgagcttgagttctgcactccagatatgtgccaaaactagtaaaacttaacggacttacaaccttgtcagtttttttaatgaggcagggatactctgtttttcacactaaacatatgaatgcagcactgctgcctcagctcagcttcgtgcctgggttccccactggtctgggaagactgttgtgctccatagagcagtgcacatctgacccagagggtgggtgttcataactgctacttgctctgctctaccatgtttaaagaaatatttggatgttaaattaactcactatggtttttcacctgggaaggaaacaaattacgtactagagggcattgactggttaaaaacttgtgtatcccgggaaggacctgcggtacaggagtcagccatgtctgtgctgtgtggaaccacctgatgacatggttaacgaggaagacgatgtgttgaccggctgccgtttgaggactttggtcacccagactagacaccttctgtgctcatgtttggaaagctgaaagggaaggacagctgtgccctcctgggagctcatgtgtccctggcgctgtgctagctttcctttacagctgtttacagacaaggcaggcctgaggcagatggccactgctcttgtgatgtttgctcagaggaatatgaacattttatttttgaaaagggatgatgtggttttttgccaggtgtttataattaatcctttaatattatggttattaacctcttaaacatgaatgaattcttgattgttttaacacagtacctaagactaatgctttctgtggacaccactgagctctgcctcaactccaccctctgcgaccggaggactatgcccctagtaactgctgtcggtgtggacgctgtgctggttctgttttctaaaggagcagaaggacaggtctctgagacaggatcgttgtccctacaggaggaacagtggccttgcttcttagacggtcttcactgtgtgttttaaaacaacaacaacaacaacaacaacaacataaaactcttttgacctgtaacttaaagatcataaacttcaggcaataatattttctgtgtaagcttttaaaattatttttggggatcatagcttgttttattttgtgctataaaattaacagtattaaatgacttatattcttagaatacatcgagtgtcttttcttaacagattagtgcctttttatttttgtattccgttttacgttactggtcccagcatcaaaacccttgtttccatggcctgtttgtatattgtctcaataaaacttgcatcagccggtggtggcggcagcaaaaaaaaaaa

(19) United States
(12) Patent Application Publication
(10) Pub. No.: US 2019/0241968 A1
(43) Pub. Date: Aug. 8, 2019

(54) BIOMARKER FOR PREDICTING RESPONSIVENESS TO ANTICANCER AGENT FOR GASTRIC CANCER AND USE THEREOF

(71) Applicant: SAMSUNG LIFE PUBLIC WELFARE FOUNDATION, Seoul (KR)

(72) Inventors: Jeeyun LEE, Seoul (KR); Kyoung Mee KIM, Seoul (KR)

(21) Appl. No.: 16/249,115

(22) Filed: Jan. 16, 2019

(30) Foreign Application Priority Data
Jan. 16, 2018 (KR) 10-2018-0005488

(51) Int. Cl. C12Q 1/6886; C12Q 1/6827; C12Q 1/6853

(52) U.S. Cl. CPC C12Q 1/6886; C12Q 1/6853; C12Q 1/6827

(57) ABSTRACT

The present disclosure relates to a biomarker for predicting responsiveness to an anticancer drug for gastric cancer and a use thereof, and more specifically, copy numbers of a human epidermal growth factor 2 (HER2) or Crk-like protein (CRKL) in DNA obtained from a HER2-positive gastric cancer patient were measured through new generation sequencing (NGS) or circulating cell-free DNA (cfDNA) analysis to demonstrate that sensitivity to the therapeutic effect of HER2-positive gastric cancer-targeting anticancer agents, specifically, the combination of capecitabine/oxaliplatin (CapeOx) and lapatinib, is significantly increased according to the copy number. Therefore, the present disclosure is expected to be effectively used for prediction of the effectiveness of the response of a subject with respect to the HER2-positive gastric cancer-targeting anticancer agent.

### CROSS-REFERENCE TO RELATED APPLICATION

This application claims priority to and the benefit of Korean Patent Application No. 10-2018-0005488, filed on Jan. 16, 2018, the disclosure of which is incorporated herein by reference in its entirety.

### FIELD

The present disclosure relates to a biomarker for predicting the responsiveness to an anticancer drug for gastric cancer and a use thereof, and more particularly, to a marker composition for predicting the response of a human epidermal growth factor 2 (HER2) positive gastric cancer-targeting anticancer agent, which includes a HER2 or Crk-like protein (CRKL) gene, or a protein encoded by the gene, a composition and a kit for predicting the response of a HER2-positive gastric cancer-targeting anticancer agent, which includes an agent for detecting mRNA or a protein of the gene, and a method of predicting the effectiveness of the response of a subject to a HER2-positive gastric cancer-targeting anticancer agent using the biomarker.

### BACKGROUND

HER2 has been known as a first validated novel target in HER2-overexpressed gastric cancer, and in this regard, in the ToGA international clinical trial, the greatest overall survival (OS) according to trastuzumab is shown in a tumor patient with high HER2 expression, identified by immunohistochemistry (IHC).

Although a trastuzumab antibody has been commercially successful, this antibody tends to be effective in only approximately 15% of HER2-overexpressed breast cancer patients. Therefore, there is an attempt to improve the prognosis of cancer patients who do not or insignificantly respond to trastuzumab through co-administration regarding to a degree of trastuzumab efficacy or efficacy spectrum (for a cancer cell line exhibiting a drug response). In addition, unfortunately, the major dose-limiting effect of trastuzumab is cardiotoxicity. Cardiac muscle cells have been known to express HER2, and it has been considered that trastuzumab-mediated cardiotoxicity is generally caused by a damage to HER2-overexpressed cardiac muscle cells, caused by binding of trastuzumab to HER2 expressed in cardiac muscle cells. Since both an anthracycline drug and an anti-HER2 antibody are associated with serious side effects (that is, cardiotoxicity), there are significant needs to optimize the established therapy, and develop new therapies that can reduce negative effects on the life quality by providing a more excellent anticancer effect in addition to causing less side effects on the heart and extending a patient's lifespan.

The pharmaceutical industry continuously pursues novel pharmacological options which are more effective, more specific or having less side effects than currently-administered drugs. An alternative to pharmacotherapy is being developed steadily due to genetic variability in human populations that cause substantial differences in the effects of a variety of the established drugs. Therefore, although a wide range of the pharmacological options are currently available, an additional therapy is always required when a patient does not respond.

Lapatinib (Tykerb), which is a dual EGFR1 and HER2 tyrosine kinase inhibitor (TKI), in combination with capecitabine/oxaliplatin (CapeOx) was tested in the Lapatinib Optimization Study in HER2-positive Gastric Cancer (LOGiC) trial, and in the Asian ErbB2+ Gastric Cancer (TyTAN) trial, both Lapatinip (Tykerb) and taxol were tested in combination with paclitaxel. However, both trials failed to demonstrate the improvement in the overall survival (OS) according to the additional use of lapatinib.

Meanwhile, gastric cancers have been known as the disease with molecular heterogeneity. As the understanding of genomic subtypes of gastric cancer deepens, it is becoming significant that HER2-overexpressed gastric cancer is associated with accompanying genetic alterations. Based on The Cancer Genome Atlas (TCGA) research, simultaneous and recurrent focal amplifications were identified in ERBB2-positive gastric cancer at CCNE1, CDK6, EGFR, MET and MYC loci, and various genomic alterations in HER2-overexpressed patients treated with trastuzumab using proteomic and high-through sequencing (HTS) technologies have been reported. However, it has not been known whether these accompanying genetic transformation affect responsiveness or resistance to HER2-targeting agents in gastric cancer.

### SUMMARY

The present disclosure is suggested to solve the above-described problems, and to this end, the inventors had measured copy numbers of HER2 and CRKL through new generation sequencing (NGS) for DNA obtained from tissue and plasma in a HER2-positive gastric cancer patient treated with the combination of CapeOx and lapatinib, which are HER2-positive gastric cancer-targeting anticancer agents, demonstrating that the sensitivity to a therapeutic effect of a target anticancer agent is significantly increased according to the copy numbers of the genes. Base on this, the present disclosure was completed.

The present disclosure is directed to providing a marker composition for predicting the response of a HER2-positive gastric cancer-targeting anticancer agent, which includes a HER2 or CRKL gene, or a protein encoded by the gene.

The present disclosure is also directed to providing a composition for predicting the response of a HER2-positive gastric cancer-targeting anticancer agent, which includes an agent for measuring an mRNA level of a HER2 or CRKL gene, or a level of a protein encoded by the gene.

The present disclosure is also directed to providing a kit for predicting the response of a HER2-positive gastric cancer-targeting anticancer agent, which includes the composition.

The present disclosure is also directed to providing a method of providing information for predicting the effectiveness of the response of a subject to a HER2-positive gastric cancer-targeting anticancer agent, the method including the following steps:

(a) extracting genomic DNA from a biological sample isolated from a HER2-positive gastric cancer patient;

(b) analyzing the copy number of a HER2 or CRKL gene of the extracted genomic DNA; and

(c) determining the patient as a subject exhibiting an effective response to the HER2-positive gastric cancer-targeting anticancer agent when the copy number of the analyzed HER2 or CRKL gene is 2 or more.

However, technical problems to be solved in the present disclosure are not limited to the above-described problems, and other problems which are not described herein will be fully understood by those of ordinary skill in the art from the following descriptions.

One aspect of the present disclosure provides a marker composition for predicting the response of a HER2-positive gastric cancer-targeting anticancer agent, which includes a HER2 or CRKL gene, or a protein encoded by the gene.

In one exemplary embodiment of the present disclosure, the HER2 gene may consist of a base sequence represented by SEQ ID NO: 1.

In another exemplary embodiment of the present disclosure, the CRKL gene may consist of a base sequence represented by SEQ ID NO: 2.

In still another exemplary embodiment of the present disclosure, the HER2-positive gastric cancer-targeting anticancer agent may be any one or more selected from the group consisting of capecitabine, oxaliplatin and lapatinib.

Another aspect of the present disclosure provides a composition for predicting the response of a HER2-positive gastric cancer-targeting anticancer agent, which includes an agent for measuring an mRNA level of a HER2 or CRKL gene, or a level of a protein encoded by the gene.

In one exemplary embodiment of the present disclosure, the agent for measuring an mRNA level of the gene may be sense and antisense primers or probes, which complimentarily bind to the mRNA of the gene.

In another exemplary embodiment of the present disclosure, the agent for measuring a protein level may be an antibody specifically binding to the protein encoded by the gene.

Still another aspect of the present disclosure provides a kit for predicting the response of a HER2-positive gastric cancer-targeting anticancer agent, which includes the composition.

Yet another aspect of the present disclosure provides a method of providing information for predicting the effectiveness of the response of a subject to a HER2-positive gastric cancer-targeting anticancer agent, the method including the following steps:

(a) extracting genomic DNA from a biological sample isolated from a HER2-positive gastric cancer patient;

(b) analyzing the copy number of a HER2 or CRKL gene of the extracted genomic DNA; and

(c) determining the patient as a subject exhibiting an effective response to the HER2-positive gastric cancer-targeting anticancer agent when the copy number of the analyzed HER2 or CRKL gene is 2 or more.

In one exemplary embodiment of the present disclosure, the biological sample may be tissue, blood, plasma or serum.

In another exemplary embodiment of the present disclosure, the biological sample may be tissue or plasma.

In still another exemplary embodiment of the present disclosure, the step (b) may be performed by NGS or circulating cell-free DNA (cfDNA) analysis.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1A is a waterfall plot summarizing a drug response.

FIG. 1B is a swimmer plot.

FIG. 1C shows the H-indices of HER2 (upper panel) and the discordance in HER2 IHC between primary and metastatic tissue specimens (lower panel), and FIG. 1D shows the H-index in correlation with % tumor reduction.

FIG. 2A shows the result of massive parallel sequencing for exons of 243 genes frequently altered in gastric cancer.

FIG. 2B shows the CCNE1 amplification in drug responders and non-responders as measured by NGS.

FIG. 2C shows the HER2 log ratio in drug responders and non-responders as measured by NGS, and FIG. 2D shows the correlation between HER2 log ratios and HER2 IHC H-index as measured by NGS.

FIG. 3A shows the CT scan results for the abdomen and pelvis of an ERBB2 and CRKL co-amplified patient.

FIG. 3B shows the expression of HER2 and CRKL by IHC in a tumor specimen.

FIG. 3C shows ERBB2 amplification and CRKL amplification in a PDC line, confirmed by qPCR.

FIG. 4 is a waterfall plot for the subset of patients with ctDNA evaluation at a baseline.

FIGS. 5A shows the results of ctDNA follow-up in lapatinib-treated GC patients in correlation with radiologic evaluation.

FIGS. 5B shows the results of ctDNA follow-up in lapatinib-treated GC patients in correlation with radiologic evaluation.

### DETAILED DESCRIPTION

Hereinafter, the present disclosure will be described in detail.

The inventors found a biomarker capable of predicting the sensitivity to the therapeutic effect of the combination of CapeOx and lapatinib from DNA obtained a HER2-positive gastric cancer patient through NGS or cfDNA analysis, and therefore, the present disclosure was completed.

Therefore, the present disclosure provides a marker composition for predicting the response of a HER2-positive gastric cancer-targeting anticancer agent, which includes a HER2 or CRKL gene, or a protein encoded by the gene.

In addition, the present disclosure provides a composition for predicting the response of a HER2-positive gastric cancer-targeting anticancer agent, which includes an agent for measuring an mRNA level of a HER2 or CRKL gene, or a level of a protein encoded by the gene, and a kit for predicting the response of a HER2-positive gastric cancer-targeting anticancer agent, which includes the composition.

The HER2 gene according to the present disclosure (Homo sapiens erb-b2 receptor tyrosine kinase 2 (ERBB2), transcript variant 1, mRNA, NCBI accession number: NM_004448) may consist of a base sequence of SEQ ID NO: 1, the CRKL gene according to the present disclosure (Homo sapiens CRK like proto-oncogene, adaptor protein (CRKL), mRNA, NCBI accession number: NM_005207) may consist of a base sequence of SEQ ID NO: 2, and homologs of each base sequence are included in the scope of the present disclosure. Specifically, each gene may include a base sequence having 70% or more, preferably 80% or more, more preferably 90% and most preferably 95% sequence homology with the base sequence of SEQ ID NO: 1 or SEQ ID NO: 2.

The “gastric cancer”, which is the target disease of the present disclosure, refers to a malignant tumor occurring in the stomach, such as gastric adenocarcinoma occurring at the gastric mucosal epithelium, a malignant lymphoma occurring at the submucosal layer, muscle sarcoma or interstitial tumor, and generally refers to gastric adenocarcinoma. In the present disclosure, gastric cancer is more preferably gastric cancer with HER2 amplification.

The term “HER2-positive gastric cancer” used herein refers to a tumor in which the copy number of the HER2 gene associated with malignancy of a tumor and prognosis in a gastric cancer patient is increased compared with the HER2 gene of a normal individual. The HER2-positive gastric cancer in which the copy number of the HER2 gene is increased and overexpressed has high possibility of the metastasis of cancer cells, has a more increased metastasis frequency in the early stage of the gastric cancer, and has poor prognosis compared with other types of gastric cancer.

The term “HER2 gene” used herein regulates the proliferation, division and repair of cells, and produces a HER2 protein having tyrosine kinase activity. The HER2 protein serves as a receptor on the cell membrane, and serves to promote the proliferation and division of cells when activated by an extracellular hormone. The HER family consists of four receptors such as an epithelial growth factor receptor (EGFR), HER2, HER3 and HER4, and forms dimers with other receptors, thereby amplifying and transmitting a transmitted signal. All the members of the HER family are associated with the proliferation of cancer cells, and particularly, HER2 is overexpressed in cancer patients with breast cancer, ovarian cancer, lung cancer, gastric cancer, uterine cancer, rectal cancer, pancreatic cancer, bladder cancer, etc., and it has been reported that an HER2 receptor is overproduced by genetic alterations to rapidly proliferate cancer cells. Particularly, the overexpressed HER2 forms homologous and heterologous dimers while not binding to a ligand, and is known to activate transcription of various cancer genes. As target therapeutic agents for overexpressing a HER2 protein, Herceptin and Tykerb are known. However, these drugs only target overexpressed HER2, and due to a resistance, they have been known to have poor prognosis of treatment.

The term “CRKL gene” used herein activates signaling pathways of RAS and JUN kinases, and encodes a protein kinase containing SH2 and SH3 (src homology) domains, which have been known to alternate fibroblasts in a RAS-dependent way. This is a substrate of a BCR-ABL tyrosine kinase, plays a critical role for BCR-ABL-induced transformation of fibroblasts, and is also known to have carcinogenic potential.

In the present disclosure, the HER2-positive gastric cancer-targeting anticancer agent may be any one or more selected from the group consisting of capecitabine, oxaliplatin and lapatinib. More preferably, the capecitabine, oxaliplatin and lapatinib are simultaneously, separately or sequentially administered, but the present disclosure is not limited thereto.

An agent for detecting mRNA of the gene may be sense and antisense primers or probes, which complimentarily bind to mRNA, but the present disclosure is not limited thereto.

The term “primer” used herein is a short gene sequence which becomes a start point of DNA synthesis, and refers to an oligonucleotide synthesized to be used in diagnosis, DNA sequencing, etc. The primers may be used by being synthesized conventionally in a length of 15 to 30 base pairs, which may be different according to the purpose of a use, and may be altered through methylation or capping by a known method.

The term “probe” used herein refers to a nucleic acid which can specifically bind to mRNA, has a length of several to hundreds of base pairs, and is manufactured through enzymatic/chemical isolation and purification or synthesis. The presence of mRNA may be confirmed by labeling with a radioisotope, enzyme or phosphor, and the probes may be designed and modified by a known method.

The agent for detecting a protein may be an antibody specifically binding to a protein encoded by the gene, but the present disclosure is not limited thereto.

The term “antibody” used herein includes an immunoglobulin molecule having immunological responsiveness to a specific antigen, and includes both a monoclonal antibody and a polyclonal antibody. In addition, the antibody includes forms produced by genetic engineering such as a chimeric antibody (e.g., a humanized murine antibody) and a heterologous antibody (e.g, a bispecific antibody).

A kit for predicting the response of an anticancer agent of the present disclosure may consist of a composition, solution or device, which consists of one type or more components, which are suitable for an analysis method.

In an exemplary embodiment of the present disclosure, drug efficiency was tested by combination administration of CapeOx and lapatinib to a patient diagnosed with gastric cancer (see Example 3), and HER2 staining was performed to confirm the relationship between the drug treatment and HER2 amplification, showing that there is a significant correlation between tumor reduction by CapeOx and lapatinib and high H-score (see Example 4).

In another exemplary embodiment of the present disclosure, the alteration of the copy number in a HER2-positive tumor was confirmed through NGS (see Example 5), and when CRKL knockdown was performed in HER2-amplified PDC by establishing a PDC line from a patient with ERBB2 and CRKL amplifications to investigate a mechanism of de novo resistance to HER2 targeted treatment, it was statistically confirmed that a resistance to lapatinib is overcome (see Example 6). In addition, a HER2-amplified patient and a patient converted into HER2-negative tumor were confirmed by performing IHC using biopsy tissue from a primary lesion in a patient treated with CapeOx/lapatinib (see Example 7), and responsiveness to treatment according to ERBB2 and CRKL amplifications based on cfDNA NGS was confirmed from the peripheral blood of a patient (see Example 8).

The present disclosure also provides a method of providing information for predicting the effectiveness of the response of a subject to a HER2-positive gastric cancer-targeting anticancer agent, the method including the following steps:

(a) extracting genomic DNA from a biological sample isolated from a HER2-positive gastric cancer patient;

(b) analyzing the copy number of a HER2 or CRKL gene of the extracted genomic DNA; and

(c) determining the patient as a subject exhibiting an effective response to the HER2-positive gastric cancer-targeting anticancer agent when the copy number of the analyzed HER2 or CRKL gene is 2 or more.

In the present disclosure, the step (a) is a step of extracting genomic DNA from a biological sample. More specifically, first, the subject is a HER2-positive gastric cancer patient, and genomic DNA is extracted from a biological sample of the subject.

In the present disclosure, the biological sample may be tissue, blood, plasma or serum, and preferably, tissue or plasma, but the present disclosure is not limited thereto as long as the biological sample is derived from a gastric cancer patient and a biomarker for predicting the response to an anticancer agent according to the present disclosure can be detected from the biological sample.

Here, most preferably, the biomarker HER2 according to the present disclosure can be used as a marker for predicting responsiveness to treatment when amplified in both tissue and plasma, and CRKL may be used as a marker for predicting responsiveness to treatment when amplified in tissue, but the present disclosure is not limited thereto.

In the step (b) of the present disclosure, the copy number of the HER2 or CRKL gene in the DNA extracted from the step (a) is analyzed. Here, the analysis of the copy number of the gene may be performed by various methods known in the art, and preferably, NGS or cfDNA analysis.

The step (c) of the present disclosure is a step for evaluating effectiveness of the response to the HER2-positive gastric cancer-targeting anticancer agent. More specifically, when the copy number of the HER2 or CRKL gene analyzed in the step (b) is 2 or more, the subject is a subject showing an effective response to the HER2-positive gastric cancer-targeting anticancer agent, and when the copy number of the HER2 or CRKL gene is less than 2, the subject is determined as a subject not showing an effective response to the HER2-positive gastric cancer-targeting anticancer agent.

The “effectiveness of the response to treatment” means whether or not a specific drug takes effect on cancer of each patient.

For example, the specific drug is mainly an anticancer agent, and the efficacy of such an anticancer agent may depend on the type of cancer. It also has been known that the efficacy of an anticancer agent, even though having been recognized to be effective on a certain type of cancer, can depend on a patient. The ability of an anticancer agent to take effect on cancer of each patient is referred to as the sensitivity to the anticancer agent. Therefore, when a patient expected to have sensitivity to treatment (responder) and a patient not expected to have sensitivity to treatment (non-responder) can be predicted before the initiation of treatment according to the present disclosure, a chemotherapy with high effectiveness and safety may be realized. The anticancer agent of the present disclosure is a HER2-targeted anticancer agent, and it is selected from the group consisting of capecitabine, oxaliplatin, lapatinib and a salt thereof.

The term “prediction” used herein is used to refer to the possibility of a target patient advantageously or disadvantageously responding to a drug or a drug set. In one aspect, prediction relates to a degree of such a reaction. For example, prediction refers to whether the patient will survive without recurrence of cancer after treatment, for example, treatment with a specific therapeutic agent, surgical removal of a primary tumor and/or chemotherapy during a specific period, and/or to the probability of survival. The prediction in the present disclosure may be clinically used to determine treatment by selecting a most suitable treatment method for a cancer patient. The prediction of the present disclosure is a useful tool for predicting whether a patient will advantageously respond to treatment, for example, given treatment such as the administration of a given therapeutic agent or composition, surgical intervention, chemotherapy, or will be survived for a long time after treatment.

Hereinafter, to help in understanding the present disclosure, exemplary examples will be suggested. However, the following examples are merely provided to promote understanding of the present disclosure, and not to limit the present disclosure.

### EXAMPLES

### Example 1. Preparation and Method of Experiment

### 1-1. Selection of Patient Group

Patients participating in the experiment are patients with histologically confirmed metastatic and/or recurrent gastric adenocarcinoma, which is potentially resectable, and among primary or metastatic tumor tissues, HER2 was considered positive by IHC 3+, or IHC 2+ with ERBB2 gene amplification by silver in situ hybridization (SISH).

The trial was conducted in accordance with the Declaration of Helsinki and the Guidelines for Good Clinical Practice (GCP, ClinicalTrial.gov.Identifier: NCT#02015169). The trial protocol was approved by the institutional review board of Samsung Medical Center (Seoul, Korea), and all patients provided written informed consent before enrollment to participate in the experiment. Meanwhile, lapatinib was provided by GlaxoSmithKline (GSK) and Novartis (Seoul, Korea), which, however, were not involved in patient recruitment, data analysis, or manuscript preparation.

The patients participating in this trial were at least 18 years old, and had at least one measurable lesion according to Response Evaluation Criteria in Solid Tumors (RECIST 1.1) or had an Eastern Cooperative Oncology Group (ECOG) status of 0 or 1. Meanwhile, patients excluded from this trial were patients who had been previously treated with radiotherapy, palliative chemotherapy or investigational therapy.

In addition, potentially resectable patients with liver metastases were limited to those with 2 to 5 occurrences of liver metastases, and the patients suspicious of peritoneal seeding by imaging without solid evidence of ascites and/or peritoneal enhancement were allowed to participate in the experiment at the discretion of an investigator.

### 1-2. Experiment Design and Treatment

The preliminary open-label trial was designed as a single-arm phase II test at an academic cancer center, and the enrolled patients were administered with CapeOx (capecitabine 1,700 mg/m²+oxaliplatin 130 mg/m²) and lapatinib (1,250 mg/m²). More specifically, drug treatment was performed in 21-day cycles, consisting of intravenous injection with oxaliplatin on day 1 (for up to 8 cycles) and oral administration of capecitabine two times a day from day 1 to day 14. Here, lapatinib was orally administered continuously up to 8 cycles. When patients exhibited partial response (PR) or complete response (CR) during treatment, they were subjected to evaluation for the possibility of curative resection. In this case, patients were subjected to curable resection if possible, or otherwise subjected to drug treatment for up to 8 cycles.

### 1-3. Evaluation

The medical history, physical examination, blood tests, urinalysis, electrocardiography, echocardiogram, chest X-ray, and abdomen and pelvis CT scan results of the patients were reviewed. The physical examination, chest X-ray, and blood tests were repeatedly performed before starting each cycle of chemotherapy. Tumor responses were evaluated for every two cycles according to the RECIST 1.1 criteria, and toxicities were graded based on the National Cancer Institute's Common Terminology Criteria for Adverse Events (CTCAE) 4.0.

### 1-4. Sampling of Tumors

Tumor tissues were selectively collected from patients according to the progression of a disease, and plasma DNA was collected at baseline and all CT scan evaluations and disease progressions. In this case, all of baseline tumor tissue specimens were archival tissue specimens prior to chemotherapy.

### 1-5. Determination of MSI Status and in situ Hybridization for EBV

To evaluate gastric tumor tissues in terms of an Epstein-Barr Virus (EBV) status and microsatellite instability (MSI), IHC was performed on formalin-fixed and paraffin-embedded (FFPE) tissue sections using an anti-MLH1 antibody (ES05 clone; 1:100 dilution, Novocastra, UK). In addition, for MSI analysis, as described above, five markers having mononucleotide repeats (BAT-25, BAT-26, NR-21, NR-24, and NR-27) were studied. Each sense primer was end-labeled with FAM, HEX or NED, and Pentaplex PCR was performed to analyze an amplified PCR product using an Applied Biosystems PRISM 3130 automated genetic analyzer, and an allelic size was estimated using Genescan 2.1 software (Applied Biosystems, Foster City, La.). Here, a sample in which an allelic size was changed at three or more microsatellites was considered MSI-H.

In addition, to evaluate the EBV infection status, EBER in situ hybridization was performed. The IHC samples were subjected to semi-quantitative analysis for HER2 using H-score. Specifically, H-score is a value obtained by multiplying the ratio of stained tumor cells by the intensity of staining (0=none, 1=1+, 2=2+ and 3=3+). Here, the H-score is in the range from 0 (no staining at tumor) to 300 (strong staining throughout the tumor).

### 1-6. Blood Samples and Isolation and Quantification of cfDNA

To compare mutation profiles in pre-treatment and sequential therapy in subgroups of HER2-positive patients, genetic alterations were analyzed in cfDNA with ERBB2 (HER2) amplification using a target 73-gene cfDNA NGS assay (Guardant360®).

More specifically, as described above, in this method, 5 to 30 ng of DNA was isolated from plasma through molecular barcoding, NGS using Illumina Hi-Seq 2500 platform, and a proprietary bioinformatics pipeline of the CLIA-certified, CAP-certified laboratory (Guardant Health, Inc., Redwood City, Calif.), and then target hybrid capture was performed.

In addition, regarding ERBB2 (HER2) amplification, the absolute copy number in plasma was first reported, and it is a function of a degree of tumor DNA shedding and the copy number in tumor tissue. Since most cfDNA is derived from a white blood cell (germline) and has a copy number of 2.0, it makes a small contribution to a copy number of circulating tumor DNA, and a small increase in a gene copy number in plasma has been known to reflect a much higher copy number in tumors. The absolute plasma copy number of 2.4 or more is marked as ++, indicating the 50^{th} percentile or above with respect to the amplification of ERBB2 (HER2) copy number in the Guardant360 database, and is known to predict an objective response to HER2-targeted treatment with respect to advanced gastric cancer, breast cancer and anticancer agent and disease control. The plasma copy number of 4.0 or more is marked as +++, indicating the 90^{th} percentile or above.

### 1-7. Patient-Derived Cell Culture

A fresh tissue sample was washed with serum-free RPMI 1640, cut finely, and enzymatically dissociated at 37 ° C. for 2 hours with stirring in serum-free RPMI 1640 containing 0.4 mg/ml collagenase (Gibco, Carlsbad, Calif., USA), 0.5 mg/ml dispase (Gibco), and 0.2 mg/ml DNase I (Roche, Mannheim, Germany). Afterward, the cells were cultured in 10% fetal bovine serum (FBS)-containing RPMI 1640, and at this time, the experiment was performed within four passages after PDC induction.

### 1-8. Tissue Genome Analysis

FFPE specimens of gastric cancer and matched normal mucosa containing 40% or more tumor cellularity were analyzed in detail under an optical microscope using 4 μm-thick unstained sections (10 to 20 slides) by comparison with hematoxylin & eosin-stained slides. Specifically, DNA was extracted according to standard procedures (Qiagen), and the extracted genomic DNA was dissected to 150 to 200 bps using a Covaris S220 ultrasonicator (Covaris, Woburn, Mas., USA). The extracted DNA was subjected to massive parallel sequencing for exons of 243 genes commonly altered in gastric cancer.

The MuTect algorithm was used to identify somatic mutations, and Variant Effect Predictor (VEP) was used to extract biological information from the called somatic mutations. Here, mutations detected from normal tissues mostly exhibited germ cell variants, and therefore, they were excluded from further analysis. The alteration of a copy number was identified using RobustCNV, and the read depth at informative capture targets in tumor samples was calibrated using depths observed in a panel of normal (non-cancer) diploid genomes to estimate a copy ratio. The resulting copy-ratio profiles were segmented using the circular binary segmentation (CBS) algorithm, and the segments were assigned gain, loss or normal copy calls using a cutoff derived from a parameter in a segment of a normalized mapping depth and a tuning parameter which was set based on the comparison to array-CGH calls in a separate validation experiment.

### 1-9. Sample Size and Statistical Analysis

According to single-arm binomial design, a sample size of 29 patients was needed to accept the hypothesis that true CR is greater than 20% with 80% power and to reject the hypothesis that CR is less than 20% with 1-sided alpha of 5%, and by including 10% non-measurable patients, the target sample size was 32 patients.

Meanwhile, the primary evaluation item of the trial was a CR rate according to RECIST, which includes both radiologic and pathologic CRs, and the secondary evaluation items were a response rate (RR), a disease control rate (DCR), a progression-free survival (PFS), an overall survival (OS), a safety profile, and exploratory biomarker analysis. The PFS was defined as the time from the start of treatment to the date of disease progression or death, and the OS was defined as the date from the start of treatment to the data of death from any cause. The RR was calculated as the percentage of patients experiencing CR or RR, and DCR as RR+ stable disease (SD), confirmed according to RECIST 1.1 guidelines.

### Example 2. Clinicopathological Characteristics of Patients

Thirty-two patients were enrolled in this research between May of 2013 and November of 2015, and the clinicopathological characteristics of the 32 enrolled patients are shown in Table 1 below.

TABLE 1

| | | N = 32 | % |
|---|---|---|---|
| Sex | Male | 26 | 81.3 |
| | Female | 6 | 18.8 |
| Age | Range | 23-80 | |
| | Median | 64 | |
| ECOG PS | 0 | 1 | 3.1 |
| | 1 | 31 | 96.9 |
| Surgery | Total | 20 | 62.5 |
| | Curative intent | 14 | 43.8 |
| | TG (total gastrectomy) | 7 | |
| | STG (subtotal gastrectomy) | 6 | |
| | ESD | 1 | |
| | Palliative | 4 | 12.5 |
| | TG | 1 | |
| | STG | 1 | |
| | Gastrojejunostomy | 2 | |
| | O & C | 2 | 6.3 |
| Pathologic subtype | Adenocarcinoma | 28 | 87.5 |
| | Papillary adenocarcinoma | 3 | 9.4 |
| | Signet ring cell | 1 | 3.1 |
| Differentiation | W/D (well differentiated) | 1 | 3.1 |
| | M/D (modestly differentiated) | 16 | 46.9 |
| | P/D (poorly differentiated) | 13 | 34.4 |
| | Unknown | 2 | 6.3 |
| Lauren | | | |
| | Diffuse | 3 | 9.4 |
| | Intestinal | 6 | 18.8 |
| | Mixed | 1 | 3.1 |
| | Not determined | 22 | 68.8 |
| EBV | | | |
| | Postitive | 2 | 6.3 |
| | Negative | 18 | 56.3 |
| | Not determined | 12 | 37.5 |
| MSS | | | |
| | MSS | 29 | 90.6 |
| | MSI-High | 0 | — |
| | Not determined | 3 | 9.4 |
| HER2 overexpression | | | |
| | 2+* | 4 | 12.5 |
| | 3+ | 28 | 87.5 |

* [symbol] [symbol]
indicates data missing or illegible when filed

As shown in Table 1, the average age of the patients was 64 years old (range of 23 to 80 years old), and most of the patients were male (81.3%). In addition, 81.3% of the patients had poorly differentiated or moderately differentiated adenocarcinoma, and 3.1% of the patients had signet ring cell carcinoma. Here, it was confirmed that two patients had EBV+ tumors, and none of the patients had an MSI-H tumor. In addition, twenty-eight patients had HER2-overexpressed gastric cancer with IHC3+, and four patients (patient #29, #13, #22, and #5) were HER2-positive (IHC2+ and SISH+).

### Example 3. Confirmation of Drug Efficacy

The end date for outcome analysis was Apr. 1st of 2017, and the average follow-up time was 22.9 months, response evaluation of only 29 of the enrolled patients was valid, CR was achieved in 7 patients (21.8%; 95%, Cl, 7.5-36.1) including two patients with pathologic CRs, and thus the CR rate met the primary evaluation criteria of this research.

In addition, 15 partial responses (PRs; 46.8%) and four patients with a stable disease were observed, and overall PR was 68.8% (95% Cl, 49.9-83.8) and DCR was 81.3% (95% Cl, 63.6-92.8). Here, among the 22 patients with CR or PR, 2 patients with CR received R0 resection, one patient received radiofrequency ablation (RFA) due to liver metastasis, and two patients refused the resection of a resectable lesion after chemotherapy. Pathological CR was confirmed from post-operative specimens of two patients who received the resection.

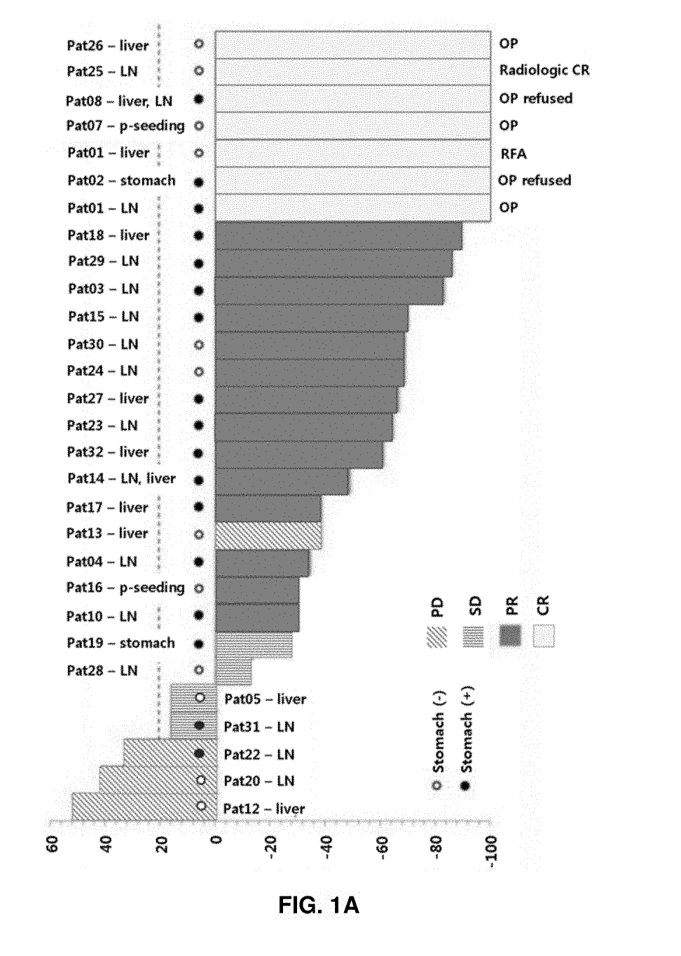

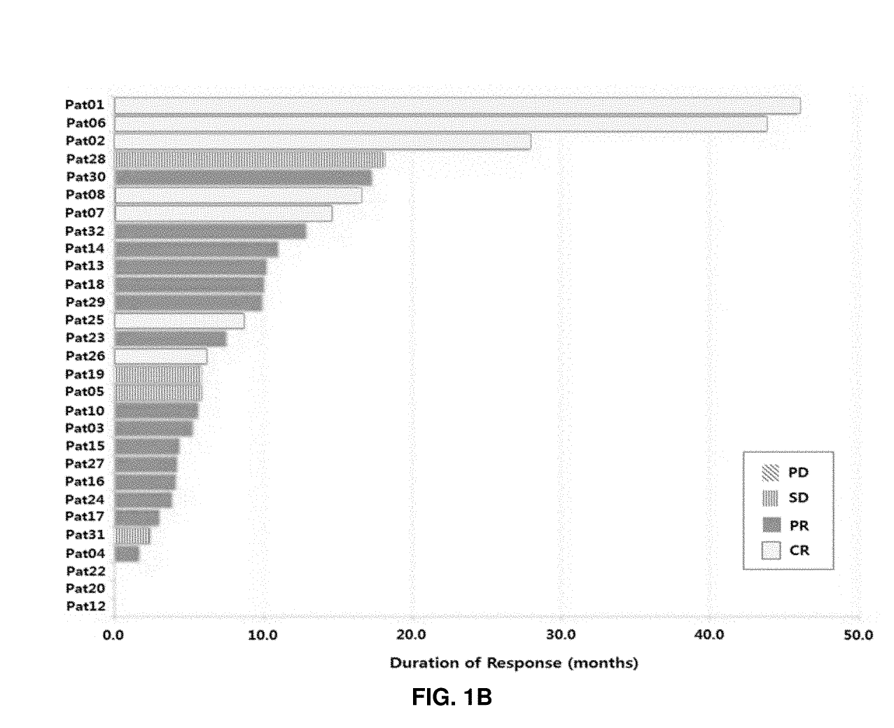

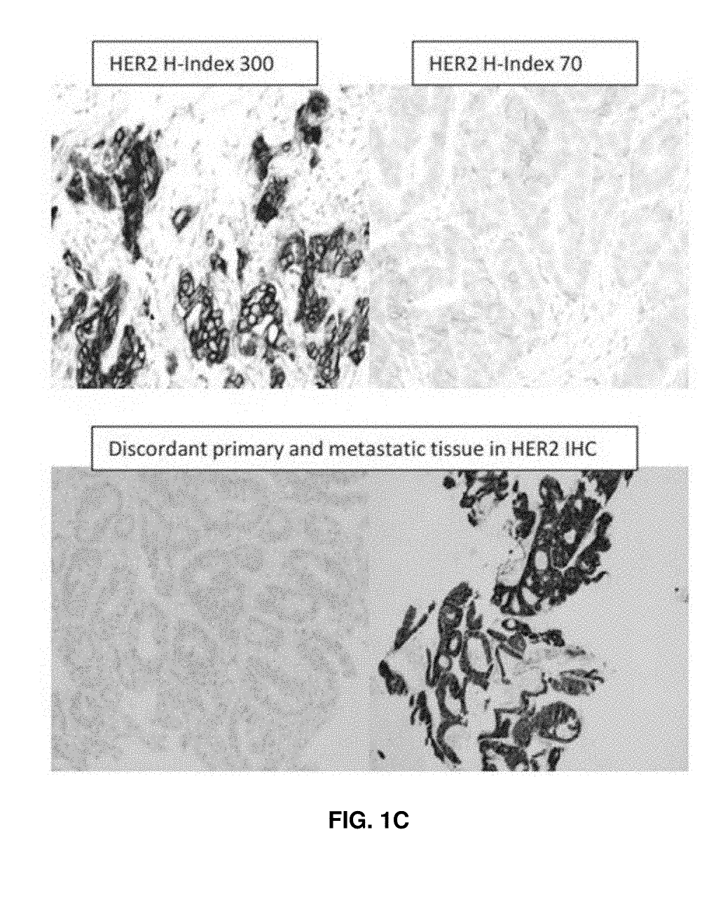

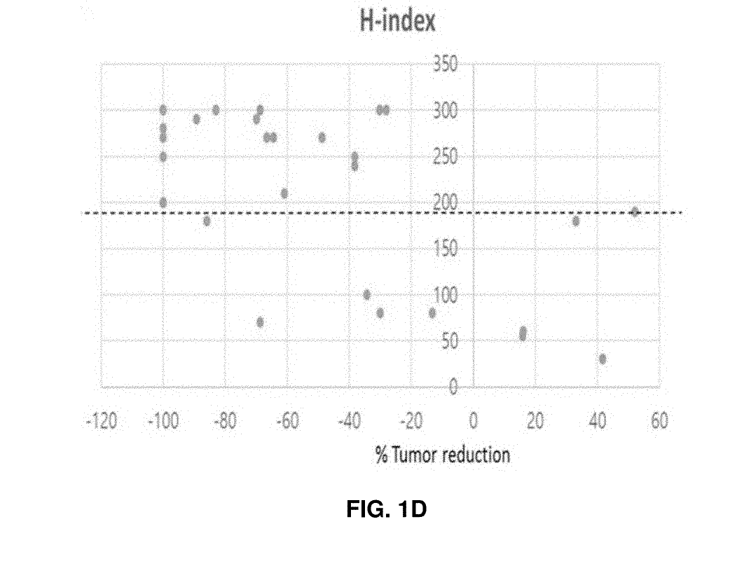

The average PFS was 9.0 months (95% Cl, 4.4-13.6 months), and the average OS was 14.2 months (95% Cl, 12.3-16.1 months). The waterfall plot and swimmer's plot of responders in FIGS. 1A and 1B show that all CR patients showed durable responses ranging from 6.2 to 45.9 months, and 9 of 14 PR patients experienced a 50% or more decrease in tumor size according to RECIST 1.1.

### Example 4. Confirmation of Inter-Tumor Heterogeneity, Heterogeneity of HER2 Staining, H-score and Tesponse to Lapatinib

From 32 patients, 10 primary-metastasis paired samples were obtained, and of the 10 paired samples, 6 patients showed concordant HER2 results, and 4 patients showed discordant HER2-positive responses between primary and metastasis (see FIG. 1C and Table 2). Here, among the 6 concordant patients, 4 patients achieved PR, 1 patient achieved CR, and 1 patient achieved PD (see Table 2).

All of the six patients showing the concordant HER2 results had synchronous metastases. Among the discordant patients, patient #22 was a HER2 2+ gastric cancer patient, but had synchronous SCN LN HER2 0, and was found to have progressive disease (PD) after 1 cycle. Patients #13 and #3 had HER2-negative primary gastric cancer tumors, which, however, recurred within 2 to 3 years after gastrectomy, and had HER2-positive liver metastases, wherein the patient #13 having heterogenous HER2 2+ liver metastasis had de novo resistance, but the patient #3 having homogenous HER2 3+ liver metastasis achieved PR due to a 80% or more decrease in tumor.

Among the 32 patients, 29 base tumor specimens were analyzed for HER2 heterogeneity staining and H-score, and these variables were correlated with response to lapatinib. As a result, among the seven PD and SD patients, 5 patients showed heterogeneity in HER2 staining, and 2 patients showed homogeneous HER2 staining. In contrast, among the 7 CR patients, 5 patients showed homogenous HER2 staining. In this case, all patients except 2 patients (metachronous liver metastasis specimens) were based on HER2 staining in primary gastric cancer, and it was confirmed that the % tumor reduction by CapeOx/lapatinib was significantly correlated with high H-score, that is, a cut-off of 200 (FIG. 1D).

### Example 5. Confirmation of Genomic Alteration in HER2-Overexpressed Gastric Cancer

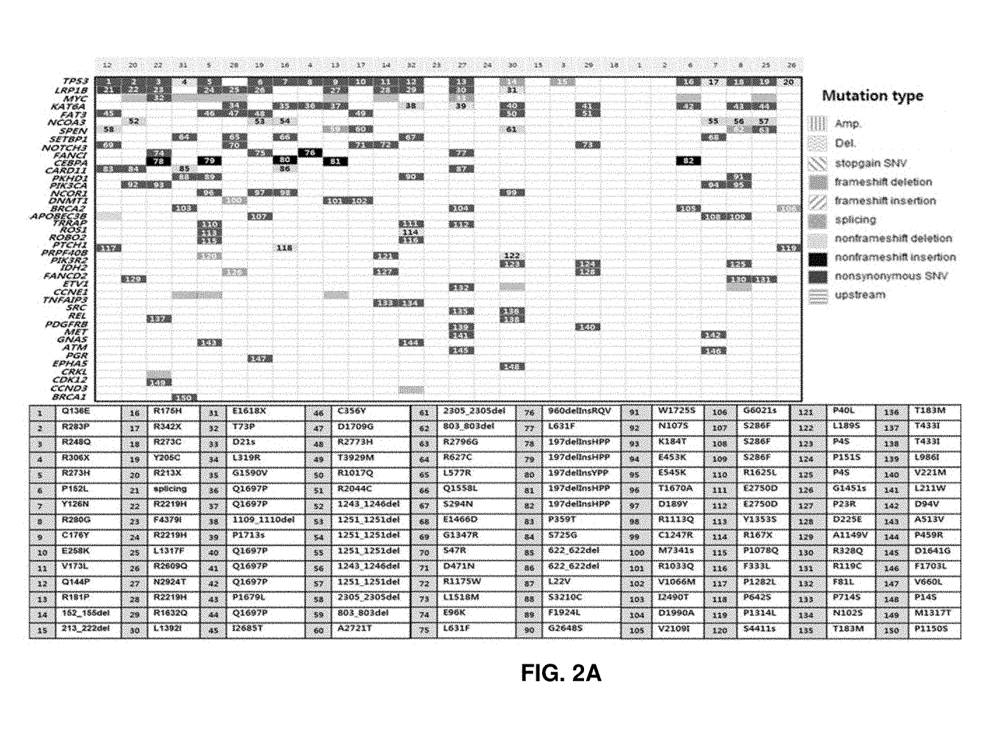

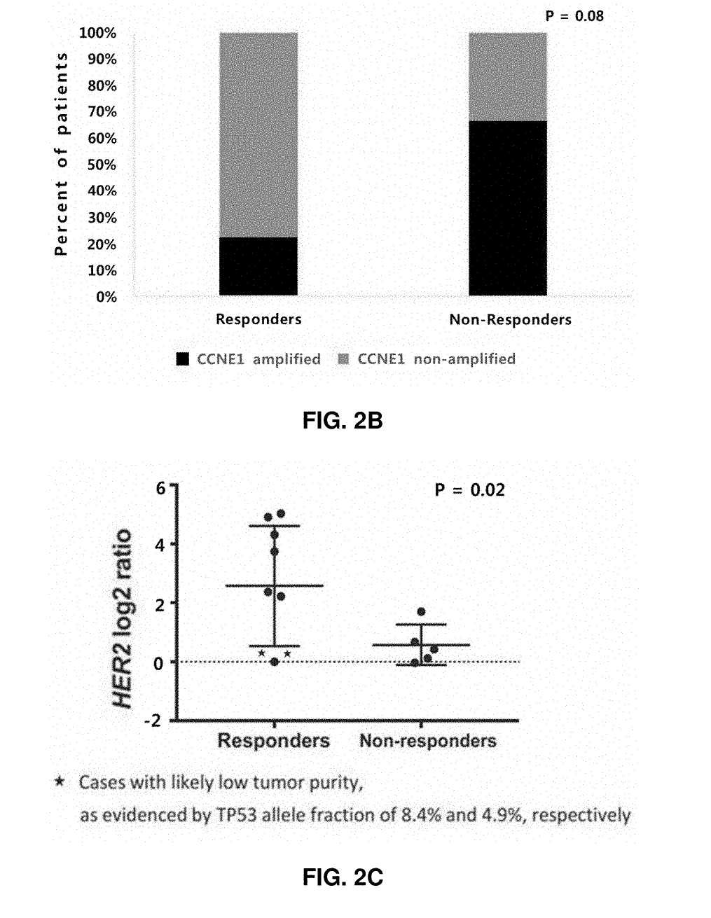

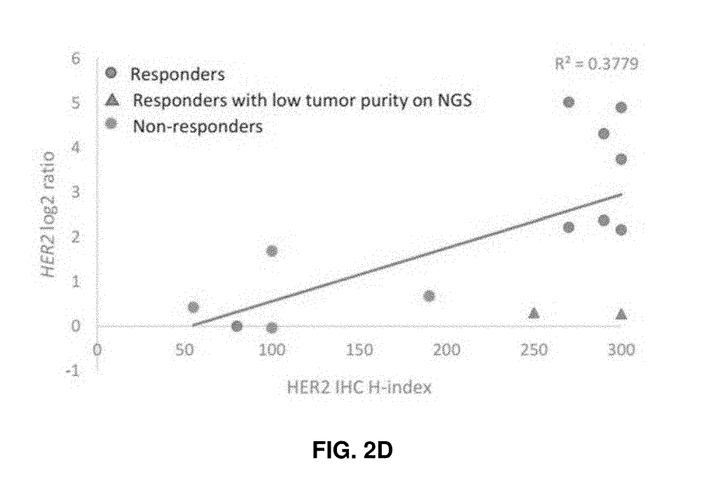

Among the 32 enrolled patients, 16 patients had sufficient tissue for NGS, and most patients' tumors (86.7%) had TP53 mutations (see FIG. 2A). In addition, 10 patients (62.5%) had at least one genomic alteration in addition to TP53. The most common alteration of a copy number was the amplification of CCNE1, which was present in 40% of HER2-positive tumors.

Interestingly, when patients with CCNE1 amplification were compared with patients without CCNE1 amplification, it was suggested that the tumor patients with CCNE1 amplification had a lower probability of responding to HER2 targeted therapy (66.7% of non-responders vs. 22.2% of responders with CCNE1 amplification; p=0.08), and it was predicted that CCNE1 amplification is a negative predictor of the response to the HER2 therapy (see FIG. 2B). In contrast, it was confirmed that, compared with patients with low-level HER2 amplification, patients with high-level HER2 amplification had a higher responsiveness to the therapy (see FIG. 2C). Among patients with PR to the therapy, two patients were confirmed to have low-level HER2 amplification based on NGS, but in these cases, it is most likely that their HER2 log2 ratio was low due to low tumor purity for an artificial reason, as proved by the low TP3 allele fractions of 8.4% and 4.9%. In addition, it was confirmed that the HER2 log2 ratio was highly correlated with HER2 IHC H-index (R²=0.378) (see FIG. 2D).

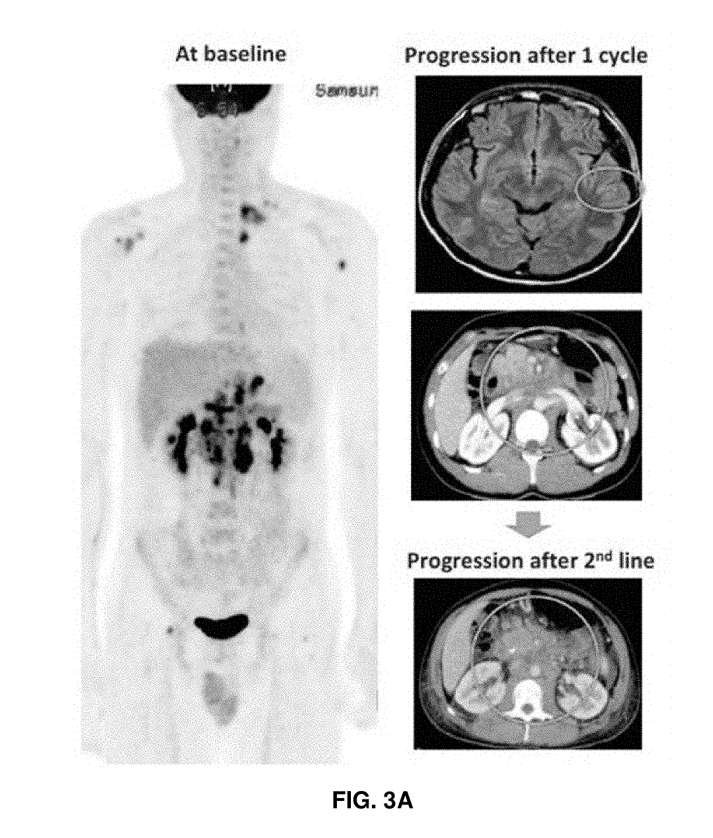

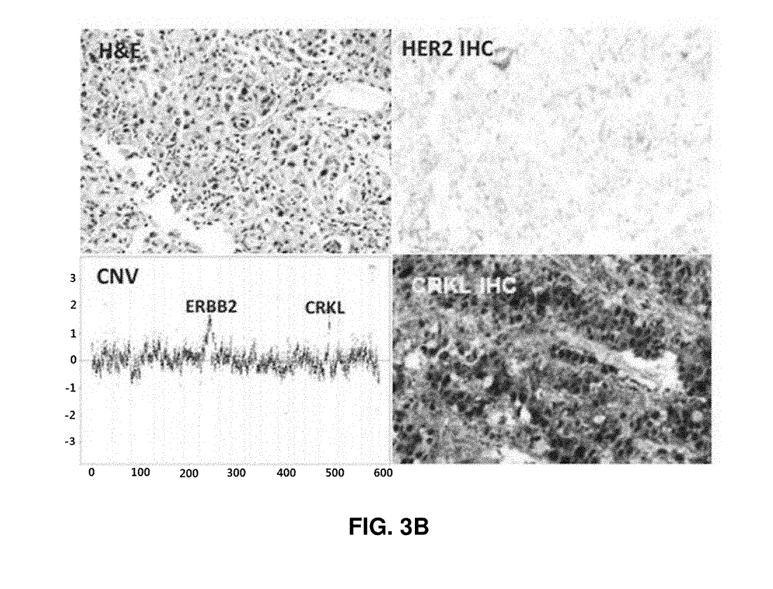

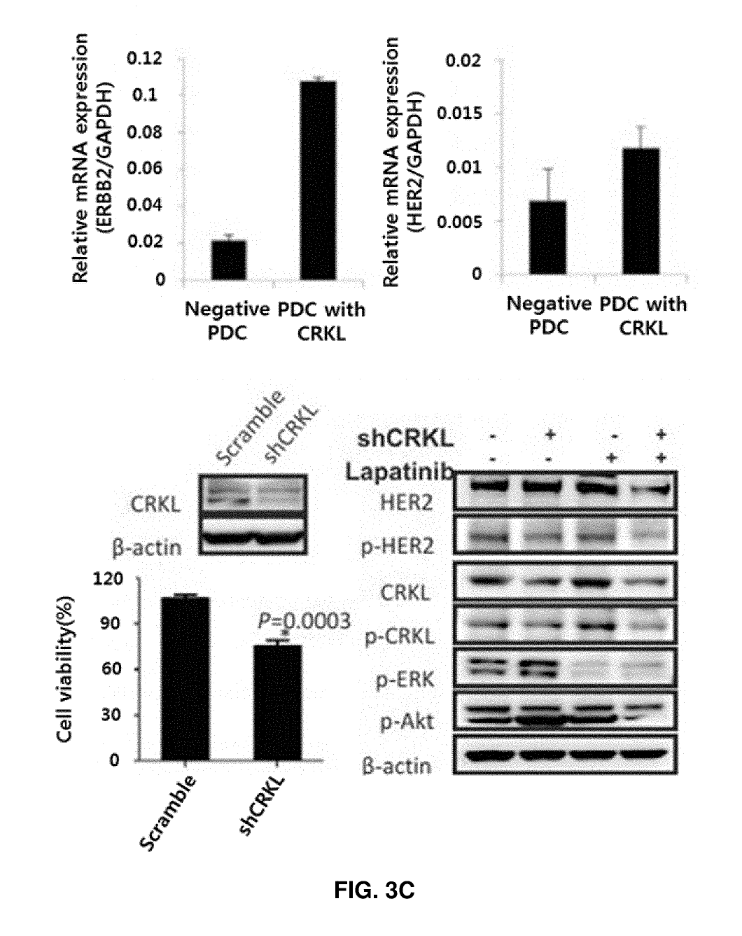

Meanwhile, a 25-year old male patient without the family history of gastric cancer was diagnosed of metastatic, HER2 amplified and CRLK amplified gastric cancer (Table 3), and the IHC result of this patient showed HER2 2+ and HER2 SISH (4.9 copies by SISH) in primary gastric tumor. The patient rapidly metastasized to the brain and abdomen after one cycle of CapeOx/lapatinib (FIG. 3A), and died of the disease after second and third cycles of chemotherapy failed. In contrast, another cancer patient with both ERBB2 and CRLK amplifications showed PR, but this patient was confirmed to have high-level HER2 amplification (log2 ratio: 4.9) and a low-level increase in CRLK (Iog2 ratio: 2.05).

TABLE 3

| | | TP53 | | | | | |
|---|---|---|---|---|---|---|---|
| | ERBB2 | allele | | Best | | Heterogeneity of | |
| Pt# | log2 | fraction | Other alterations | response | H-index | HER2 staining | CCNE1 IHC |
| 012 | 0.679 | 0.2213 | CCNE1 amp, | PD | 190 | hetero | CCNE1 IHC positive |
| | | | PTEN loss, | | | | |
| | | | NOTCH2 | | | | |
| | | | missense | | | | |
| 016 | 3.74 | 0.409 | SMAD2/4, | PR | 300 | homo | CCNE1 IHC negative |
| | | | BCL2, | | | | |
| | | | KEAP1 loss, | | | | |
| | | | RNF43 mut. | | | | |
| | | | CDK8 mut | | | | |
| 014 | 2.22 | 0.54 | Loss, | PR | 270 | homo | CCNE1 IHC negative |
| | | | APC nonsense, | | | | |
| | | | ARID1A nonsense, | | | | |
| | | | CTNNB1 | | | | |
| | | | missense, | | | | |
| | | | THOA | | | | |
| 015 | 4.31 | | | PR | 290 | homo | CCNE1 IHC negative |
| 017 | 0.307 | 0.084 | CCNE1 amp, | PR | 250 | homo | CCNE1 50% (+) IHC |
| | | | FGFR2 amp, | | | | |
| | | | IDH2 amp, | | | | |
| | | | PIK3CG missense | | | | |
| 018 | 2.37 | 0.3259 | CCNE1 amp, | PR | 290 | homo | NA |
| | | | VEGFA amp, | | | | |
| | | | CDK1NA amp, | | | | |
| | | | CCND3 amp | | | | |
| 023 | 5.02 | 0.4375 | ETV4 amp, | PR | 270 | homo | CCNE negative |
| | | | CCND3 | | | | |
| | | | frameshift | | | | |
| 024 | 4.9 | 0.4262 | CRKL amp, | PR | 300 | homo | CCNE negative |
| | | | APC nonsense | | | | |
| 005 | 0.428 | 0.2962 | CCNE1 amp, | SD | 55 | hetero | CCNE1 positive |
| | | | MYC amp, | | | | |
| | | | APC loss, | | | | |
| | | | TP53 loss, | | | | |
| | | | ARID1A | | | | |
| 019 | 1.69 | 0.1185 | CCNE1 amp, | SD | 100 | hetero | CCNE1 positive |
| | | | EGFR amp, | | | | |
| | | | SMAD3/4 loss | | | | |
| 006 | 0.281 | 0.049 | BEGFA amp, | CR | 300 | homo | NA |
| | | | ERBB3 mutation, | | | | |
| | | | ARID18 inframe | | | | |
| | | | del | | | | |
| 004 | 0.0333 | 0.62963 | SMARCA4 amp, | PD | 100 | hetero | CCNE1 negative |
| | | | NOTCH3 amp, | | | | |
| | | | BRD4 amp, | | | | |
| | | | TERT loss, | | | | |
| | | | APC frameshift | | | | |
| 013 | 0.12 | 0.2 | CCNE1 amp | PD | Stomach | hetero | CCNE1 positive |
| | | | | | HER2 | | |
| | | | | | (−) liver | | |
| | | | | | HER2 | | |
| | | | | | (3+) | | |
| 010 | | | BCL9 frameshift | PR | 80 | hetero | CCNE1 negative |
| 021 | 2.16 | 0.3789 | CDKN2A loss | NE | 300 | homo | CCNE1 negative |
| 022 | | NA | CRKL amp | PD | 180 | homo | CCNE1 negative |

### Example 6. Confirmation of Control of de novo Resistance to Lapatinib by CRKL Co-Amplification

To examine the mechanism of de novo resistance to HER2-targeted therapy, PDC line was established from patients with ERBB2 and CRKL amplifications. According to the IHC results, tumors of the patients were HER2 IHC2+, HER2 SISH-positive, and strong CRKL-positive poorly differentiated adenocarcinoma (FIG. 3B), and primary tumor and the PDC line showed similar morphologies and IHC-stained patterns (not shown). In addition, through qPCR, it was confirmed that the PDC line showed ERBB2 and CRKL amplifications (FIG. 3C).

An MTT proliferation assay demonstrated that gefitinib, lapatinib and AZD8931 (pan-HER inhibitor) do not inhibit the growth of the PDC line, and this result shows that CRKL amplification/overexpression may induce a resistance to a HER2 signal inhibitor.

Subsequently, to further characterize the significance of the CRKL amplification/overexpression in terms of the EGFR-signal inhibitor (including lapatinib), shRNA (short-hairpin RNA)-mediated CRKL knockdown was performed in HER2 amplified PDCs.

As a result, as shown in FIG. 3C, shRNA effectively inhibited CRKL expression in the CRKL-amplified/overexpressed PDCs, and CRKL knockdown in an HER2-amplified PDC line overcame lapatinib resistance in a statistically significant cell viability assay (P=0.0003; see FIG. 3C).

Likewise, the inhibition of both HER2 and CRKL by lapatinib and shCRKL demonstrated substantial ERK and AKT down-regulation as determined by western blotting (FIG. 3C, right panel).

### Example 7. Confirmation of Change of HER2 Status in Tumor Due to Disease Progression

Post-progression biopsies were possible from the primary lesions of 7 patients treated with CapeOx/lapatinib, and an IHC test for HER2 was carried out by collecting post-progression specimens of the 7 patients. More specifically, after the CapeOx/lapatinib chemotherapy (see Table 2), 4 patients (57%) exhibited continuous HER2 overexpression at the time of progression, whereas 3 patients (43%) experienced conversion to an HER2-negative tumor after chemotherapy.

### Example 8. Confirmation of Plasma ERBB2 Status as Predictor and Molecule Correlation of Response Based on cfDNA NGS

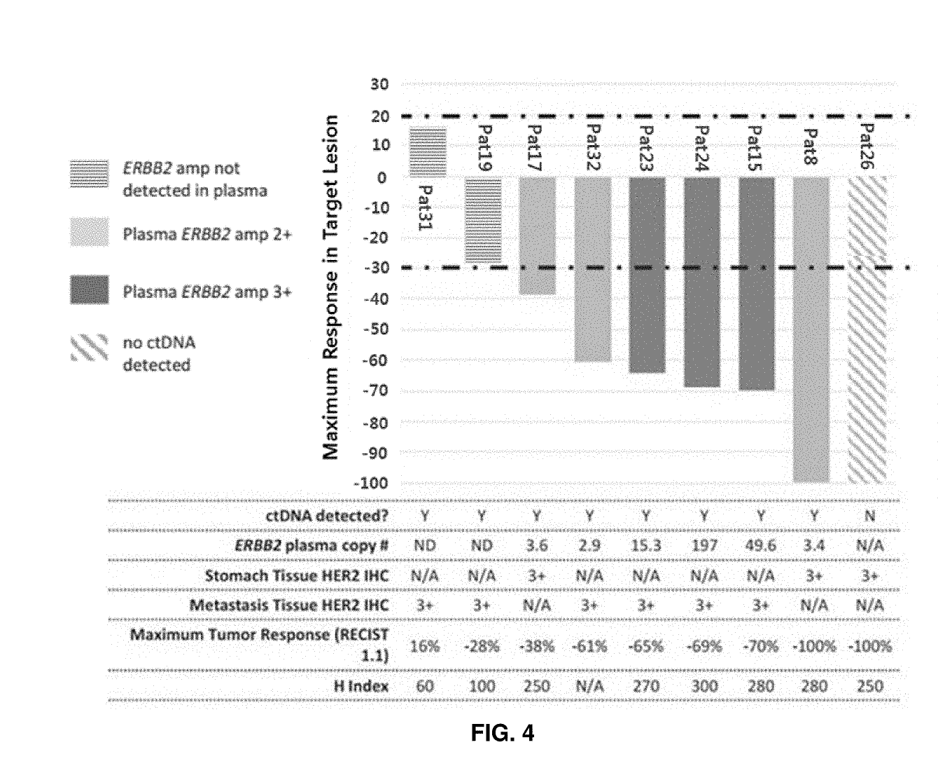

As an exploratory analysis, peripheral blood for plasma cfDNA analysis (Guardant360®) was collected from 9 of the 32 HER2-positive enrolled patients before the start of treatment and during treatment at all CT tests, one of the 9 pre-treatment samples had insufficient DNA for analysis, and the other 8 samples were suitable. Six of the 8 samples had plasma ERBB2 amplification with a response rate of 100% (95% Cl, 54-100), including one complete response (FIG. 4). There was no correlation between an ERBB2 copy number in plasma and a response depth (p-value, 0.46), and two of the 8 samples, from which ERBB2 amplification was not detected, showed a stable disease (SD). In addition, the average progression-free survival (PFS) for the six plasma ERBB2 amplified patients was 9.0 months (95% Cl, 1.8-16.2).

Meanwhile, several cases explained the correlation between the ERBB2 copy number in plasma evaluation and responsiveness to or progression of treatment.

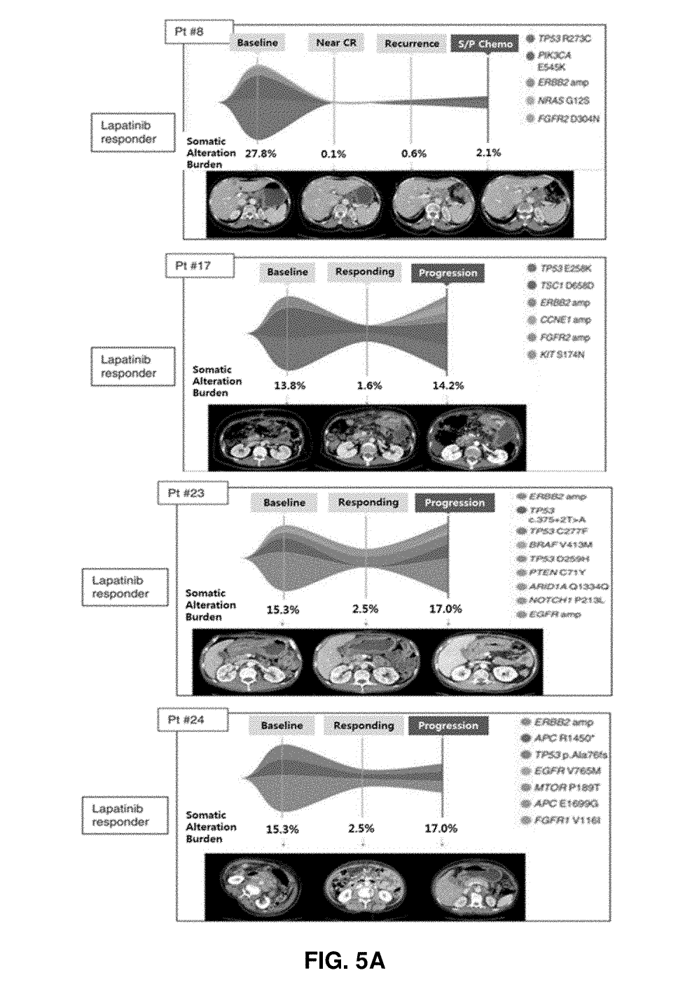

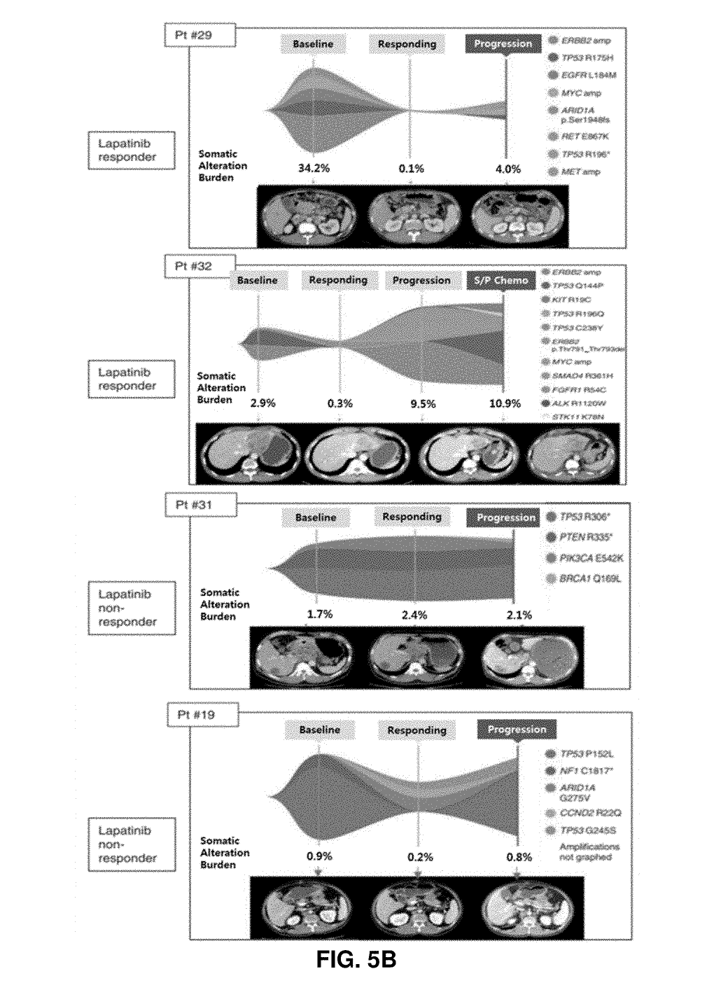

More specifically, patient #8 was diagnosed of HER2-positive gastric cancer with several liver metastasis, and at the baseline, this patient had TP53 R273C mutation having ERBB2 amplification (orange), PIK3CA E545K mutation, NRAS G12S mutation and a somatic mutation size (highest variant allele fraction at given time point) of 27.8% (FIG. 5A, Pt#8). After 8 cycles of CapeOx/lapatinib, when the patient achieved near CR (PR according to RECIST1.1), it was confirmed that the somatic alternation size was reduced to 0.1%, and the patient received resection. In addition, the cancer of the patient recurred as a soft tissue mass around the celiac axis, and cfDNA genomic profiling at this time showed recurrence of PIK3CA E545K and TP53 R273C mutations, but no HER2 amplification.

When patient #17 responded to CapeOx/lapatinib, it was confirmed that the ERBB2 amplification level was reduced. After 6 cycles of drug treatment, the patient developed peritoneal metastasis with ascites, and subsequent ctDNA showed newly emerged FGFR2 amplification and CCNE1 amplification.

During lapatinib treatment, patient #23 had EGFR amplification newly emerged, in addition to ERBB2 amplification.

Patient #24 was subjected to seven cycles of CapeOx/lapatinib treatment, and an ERBB2 amplification level was increased.

Patient #29 demonstrated multiple ctDNA genetic alterations including ERBB2 amplification, R175H mutation and MYC amplification, and at the time of the therapeutic response, the somatic mutation size was decreased from 34.2% to 0.1%, achieving CR.

After long-term maintenance of the response to lapatinib, the patient experienced recurrence of peritoneal seeding when ctDNA exhibited MYC amplification, rather than ERBB2 amplification, TP53 R175H mutation, and newly-emerged MET amplification.

At baseline, patient #32 showed ERBB2 amplification, TP53A144P mutation and TP53R196Q mutation, and after 8 cycles of CapeOx/lapatinib treatment, PR was achieved. After long-term maintenance of the response, the patient developed liver and primary tumors, and ctDNA exhibited newly-emerged MYC amplification, SMAD4 R361 H mutation and FGFR1 R54C mutation.

All of subsequent salvage chemotherapy which has correlation with the increase in somatic mutation size by ctDNA failed to work on the patient.

Interestingly, patients #31 and #19 did not have ERBB2 amplification as detected by ctDNA, and the result was confirmed through CapeOx chemotherapy.

When a HER2 or CRKL copy number in the DNA obtained from a HER2-positive gastric cancer patient was measured through NGS or cfDNA analysis, it was demonstrated that the sensitivity to the therapeutic effect of HER2-positive gastric cancer-targeting anticancer agents, specifically, the combination of CapeOx and lapatinib, is significantly increased according to the copy number. Therefore, the present disclosure is expected to be effectively used for prediction of the effectiveness of the response of a subject with respect to the HER2-positive gastric cancer-targeting anticancer agent.

It should be understood by those of ordinary skill in the art that the above description of the present disclosure is exemplary, and the exemplary embodiments disclosed herein can be easily modified into other specific forms without departing from the technical spirit or essential features of the present disclosure. Therefore, the exemplary embodiments described above should be interpreted as illustrative and not limiting in any aspect.

This application contains references to amino acid sequences and/or nucleic acid sequences which have been submitted herewith as the sequence listing text file. The aforementioned sequence listing is hereby incorporated by reference in its entirety pursuant to 37 C. F. R. § 1.52(e).

1. A method of predicting effectiveness of responsiveness of a subject to a human epidermal growth factor 2 (HER2)-positive gastric cancer-targeting anticancer agent, the method comprising:
(a) extracting genomic DNA from a biological sample isolated from a HER2-positive gastric cancer patient;
(b) analyzing the copy number of a HER2 or Crk-like protein (CRKL) gene of the extracted genomic DNA; and
(c) determining the patient as a subject exhibiting an effective responsiveness to the HER2-positive gastric cancer-targeting anticancer agent when the copy number of the analyzed HER2 or CRKL gene is 2 or more.

2. The method according to claim 1, wherein the HER2 gene consists of a base sequence represented by SEQ ID NO: 1.

3. The method according to claim 1, wherein the CRKL gene consists of a base sequence represented by SEQ ID NO: 2.

4. The method according to claim 1, wherein the HER2-positive gastric cancer-targeting anticancer agent is any one or more selected from the group consisting of capecitabine, oxaliplatin, and lapatinib.

5. The method according to claim 1, wherein the biological sample is tissue, blood, plasma, or serum.

6. The method according to claim 5, wherein the biological sample is tissue or plasma.

7. The method according to claim 1, wherein the step (b) is performed by new generation sequencing (NGS) or circulating cell-free DNA (cfDNA) analysis.

8. A kit for predicting responsiveness to a human epidermal growth factor 2 (HER2)-positive gastric cancer-targeting anticancer agent, comprising an agent for measuring an mRNA level of a HER2 or Crk-like protein (CRKL) gene, or an agent for measuring a level of a protein encoded by the gene.

9. The kit according to claim 8, wherein the agent for measuring an mRNA level of the gene is sense and antisense primers or probes, which complimentarily bind to the m RNA of the gene.

10. The kit according to claim 8, wherein the agent for measuring a protein level is an antibody specifically binding to the protein encoded by the gene.
